(19) United States
(12) Patent Application Publication
(10) Pub. No.: US 2017/0251961 A1
(43) Pub. Date: Sep. 7, 2017

(54) BIOLOGICAL INFORMATION MEASUREMENT DEVICE, MIXED VENOUS OXYGEN SATURATION ESTIMATION METHOD, AND PROGRAM

(71) Applicant: NIHON KOHDEN CORPORATION, Tokorozawa (JP)

(72) Inventors: Kota Saeki, Tokyo (JP); Naoki Kobayashi, Tokyo (JP)

(73) Assignee: NIHON KOHDEN CORPORATION, Tokorozawa (JP)

(21) Appl. No.: 15/435,133

(22) Filed: Feb. 16, 2017

(30) Foreign Application Priority Data
Mar. 4, 2016 (JP) 2016-041835

(51) Int. Cl. A61B 5/1455; A61B 5/00; A61B 5/145

(52) U.S. Cl. CPC A61B 5/14551; A61B 5/14546; A61B 5/7278

(57) ABSTRACT

Provided is a biological information measurement device, including: a blood index calculation unit which calculates at least part of a blood index associated with oxygen transport based on transmitted light or reflected light when a body part of a test subject is irradiated with light with a plurality of wavelengths; an oxygen consumption calculation unit which calculates an oxygen consumption based on a gas concentration in inspired air, a gas concentration in expired air, and a ventilation amount of the test subject; a cardiac output calculation unit which calculates a cardiac output based on a biological signal obtained by a noninvasive method from the test subject; and an SvO2 estimation unit which estimates the mixed venous oxygen saturation of the test subject by substituting the blood index, the oxygen consumption, and the cardiac output in a calculation formula.

CROSS-REFERENCE TO RELATED APPLICATION(S)

This application claims priority to and the benefit under 35 U.S.C. §119(a) of the earlier filing date of Japanese Application No. JP 2016-041835 filed Mar. 4, 2016, which is incorporated herein by reference, in its entirety, for any purpose.

### BACKGROUND

Example of the present invention relates to a biological information measurement device, a mixed venous oxygen saturation estimation method, and a program.

Mixed venous oxygen saturation (SvO2) is oxygen saturation of pulmonary arterial blood. The mixed venous oxygen saturation is used as an index of the balance between supply and demand of oxygen in the body. For example, a decrease in mixed venous oxygen saturation means a lack of oxygen supply or an increase in oxygen consumption. The mixed venous oxygen saturation changes due to respiratory failure, circulatory failure, or hypermetabolism accompanying fever or infection. Therefore, the mixed venous oxygen saturation is particularly utilized for ascertaining the state of circulation of oxygen in the whole body in the intensive care field.

In general, the mixed venous oxygen saturation is measured by inserting a Swan-Ganz catheter into the pulmonary artery. However, this method is an invasive method in which a Swan-Ganz catheter is inserted into the body of a test subject, and therefore places a large burden on the test subject.

In light of this, a method of noninvasively measuring the mixed venous oxygen saturation has been proposed. JP-T-2010-524598 (Patent Document 1) discloses a technique in which a light-emitting element and a light-receiving element are placed in the vicinity of a target deep vascular structure (for example, the pulmonary artery), and blood flowing through the deep vascular structure is irradiated with light, whereby the oxygen saturation of the blood is measured.

WO 13/112812 (Patent Document 2) discloses a system in which the mixed venous oxygen saturation is noninvasively calculated using photoacoustic imaging. In the system, a tissue to be measured is irradiated with laser light, and the mixed venous oxygen saturation is calculated using an ultrasonic wave generated accompanying light absorption.

U.S. Patent Application Publication No 2012-0065485 (Patent Document 3) discloses a device in which pulmonary arterial blood is irradiated with light using an NIRS sensor and the mixed venous oxygen saturation is measured.

In the techniques disclosed in the above Patent Documents 1 and 2, the mixed venous oxygen saturation is noninvasively measured by applying light (including laser light) from a biological tissue immediately above the pulmonary artery. In this method, in the case where light could not be applied to the exact place (the place where a blood vessel to be measured is present), the accurate mixed venous oxygen saturation may not be able to be measured. Further, in the technique disclosed in Patent Document 3, since an MRS sensor is used, light does not reach the deep region in the body, and thus, the measurement may not be able to be performed accurately in a test subject with a large physique.

That is, in the above-mentioned techniques, the mixed venous oxygen saturation may not be able to be measured accurately and noninvasively.

### SUMMARY

The invention has been made in view of the above problems, and a main object of the invention is to provide a biological information measurement device and a mixed venous oxygen saturation estimation method capable of noninvasively and accurately measuring the mixed venous oxygen saturation.

One aspect of a biological information measurement device according to the invention includes:

a blood index calculation unit which calculates at least part of a blood index associated with oxygen transport based on transmitted light or reflected light when an arbitrary body part of a test subject is irradiated with light with a plurality of wavelengths;

an oxygen consumption calculation unit which calculates an oxygen consumption based on a gas concentration in inspired air, a gas concentration in expired air, and a ventilation amount of the test subject;

a cardiac output calculation unit which calculates a cardiac output based on a biological signal obtained by a noninvasive method from the test subject; and

an SvO2 estimation unit which estimates a mixed venous oxygen saturation of the test subject by substituting the blood index, the oxygen consumption, and the cardiac output in a calculation formula derived from a principle of blood circulation and an oxygen binding amount per unit hemoglobin.

It is known that a blood index (an arterial oxygen saturation or the amount of hemoglobin in blood) associated with oxygen transport, an oxygen consumption, and a cardiac output can be noninvasively and accurately obtained by the function of a biological information monitor used in a general hospital (JP-A-8-322822 (Patent Document 4), IP-A-2005-312947 (Patent Document 5), etc.). An SvO2 estimation unit 15 estimates a mixed venous oxygen saturation (SvO2) by substituting these parameters in a calculation formula derived from the blood circulation and the oxygen transport amount per unit hemoglobin. The SvO2 estimation unit 15 uses parameters which can be noninvasively and accurately calculated in the calculation, and therefore, the mixed venous oxygen saturation (SvO2) can be continuously and accurately estimated,

The invention can provide a biological information measurement device and a mixed venous oxygen saturation estimation method capable of noninvasively and accurately measuring the mixed venous oxygen saturation.

### BRIEF DESCRIPTION OF THE DRAWINGS

FIG. 1 is a block diagram showing the configuration of a biological information measurement device 1 according to an embodiment of the present disclosure.

FIG. 2 is a view showing an example of a display screen of the biological information measurement device 1 according to an embodiment of the present disclosure.

### DETAILED DESCRIPTION

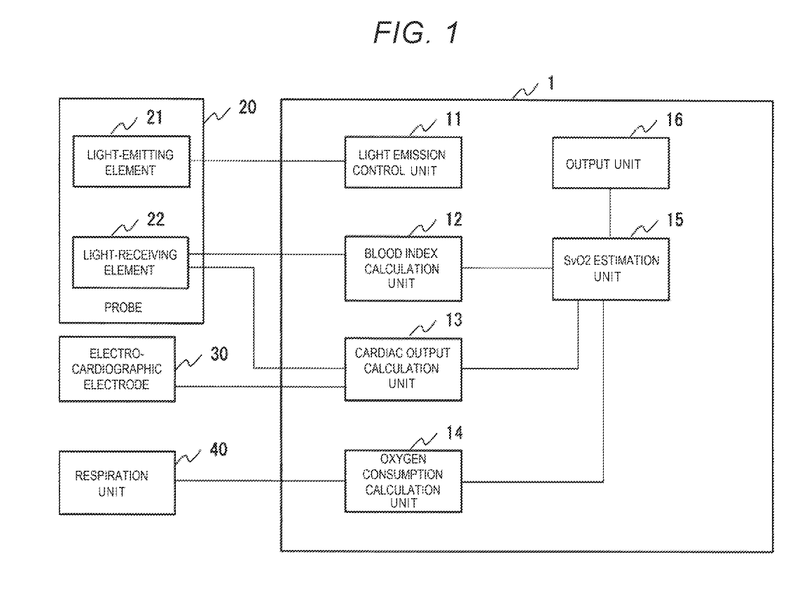

Hereinafter, embodiments of the invention will be described with reference to the drawings. FIG. 1 is a block diagram showing the configuration of a biological information measurement device 1 according to an embodiment of the present disclosure. The biological information measurement device 1 measures various vital signs (electrocardiogram, blood pressure, arterial oxygen saturation, heart rate, respiratory rate, body temperature, etc.) of a test subject. The biological information measurement device 1 may be a bed-side monitor which is used in a hospital room, or may be a medical telemeter (transmitter) which is always carried by a test subject (patient). That is, the biological information measurement device 1 may have any form or size as long as it is a device which measures the vital signs of a test subject.

The biological information measurement device 1 is connected to any type of probe, a cuff, an electrode, and the like. The biological information measurement device 1 shown in FIG. 1 is connected to a probe 20, an electrocardiographic electrode 30, and a respiration sensor 40. Incidentally, although not shown in the drawing, the biological information measurement device 1 may be connected to a cuff (air bag) for noninvasive blood pressure measurement, a body temperature sensor, or the like.

The probe 20 emits light on an arbitrary body part (for example, a fingertip, an earlobe, a forehead, or the like) of a test subject, and receives transmitted light or reflected light from the body part. The probe 20 includes a light-emitting element 21 and a light-receiving element 22. The light-emitting element 21 emits light with a plurality of wavelengths including a wavelength of light which is absorbed by water on a body part. The light-emitting element 21 emits light with a plurality of wavelengths selected from an arbitrary wavelength between, for example, 600 nm and 1300 nm on a biological tissue. The light-emitting element 21 may be any as long as it is comprised of, for example, a light-emitting diode (LED).

The light-receiving element 22 receives transmitted light obtained by transmitting the light with a plurality of wavelengths through the body part (or reflected light obtained by reflecting the light with a plurality of wavelengths from the body part). The light-receiving element 22 converts the transmitted light (or the reflected light) into an electrical signal (biological signal) and inputs the signal to a blood index calculation unit 12 and a cardiac output calculation unit 13. Although not shown in the drawing, the light-receiving element 22 may include an A/D (analog-digital) converter which performs A/D conversion of the electrical signal as appropriate. The light-receiving element 22 may be any as long as it is comprised of, for example, a photodiode.

The electrocardiographic electrode 30 includes a plurality of electrodes (a disposable electrode and a clip electrode) for measuring an electrocardiogram (ECG) of a test subject. The electrocardiographic electrode 30 is attached to a predetermined place of the chest or the limb of a test subject. The electrocardiographic electrode 30 and the biological information measurement device 1 are connected to each other through an electrical cable. The electrocardiographic electrode 30 inputs a biological signal obtained from the predetermined place of the test subject to the cardiac output calculation unit 13.

The respiration sensor 40 measures a gas concentration in expired air, a gas concentration in inspired air, and a ventilation amount of a test subject. The respiration sensor 40 may be actually comprised of a plurality of sensor devices. For example, the respiration sensor 40 may be comprised of an artificial respirator which can be connected to the so-called biological information measurement device 1 and a gas sensor and a flow sensor which can be attached to the artificial respirator. A medical worker places the gas sensor and the flow sensor on an expired air-side respiratory circuit and an inspired air-side respiratory circuit or an exhaust port of the artificial respirator. The gas sensor obtained the expired air and inspired air of the test subject from the respective respiratory circuits or the exhaust port and measures a gas concentration in the expired air and a gas concentration in the inspired air. Further, the flow sensor measures a ventilation amount of the test subject.

Further, the respiration sensor 40 may measure a gas concentration in the expired air or a gas concentration in the inspired air by a so-called side-stream system. The respiration sensor 40 is comprised of, for example, an artificial nose attached to a test subject, an adapter for sampling, a sampling tube, a gas sensor, a flow sensor to be attached to the mouth, and the like. The expired air of a test subject is sucked through the sampling tube, and the sucked gas is measured by the gas sensor. By doing this, a gas concentration in the expired air and a gas concentration in the inspired air of the test subject are measured. The flow sensor attached to the mouth measures a ventilation amount of the test subject.

Further, the respiration sensor 40 may be comprised of a gas sensor and a flow sensor attached to the mouth. The gas sensor in this configuration measures a gas concentration in expired air and a gas concentration in inspired air of a test subject. The flow sensor measures a ventilation amount of the test subject.

That is, the respiration sensor 40 may have any configuration as long as it can measure a gas concentration in expired air, a gas concentration in inspired air, and a ventilation amount of a test subject. The respiration sensor 40 inputs the measured gas concentration in the expired air, gas concentration in the inspired air, and ventilation amount of the test subject to an oxygen consumption calculation unit 14.

Subsequently, the configuration of the biological information measurement device 1 will be described. The biological information measurement device 1 includes a light emission control unit 11, a blood index calculation unit 12, a cardiac output calculation unit 13, an oxygen consumption calculation unit 14, an SvO2 estimation unit 15, and an output unit 16. Although not shown in the drawing, the biological information measurement device 1 includes a memory unit (including a primary memory device and a secondary memory device) which stores any type of data.

The light emission control unit 11 controls light with a plurality of wavelengths emitted on a body part of a test subject. Specifically, the light emission control unit 11 controls the wavelength, emission timing, and emission intensity of light emitted by the light-emitting element 21. Here, it is preferred that the light emission control unit 11 controls emission of light so that light with a plurality of wavelengths including light with a wavelength which is absorbed by water is emitted in order for the below-mentioned blood index calculation unit 12 to calculate the amount of hemoglobin in blood.

The blood index calculation unit 12 calculates at least part of a blood index associated with oxygen transport based on light (transmitted light or reflected light) obtained by transmitting or reflecting the light with a plurality of wavelengths including a wavelength of light which is absorbed by water through or from an arbitrary body part of a test subject. More specifically, the blood index calculation unit 12 calculates at least an arterial oxygen saturation, and preferably also calculates the amount of hemoglobin in blood.

The blood index calculation unit 12 obtained transmitted light or reflected light when red light (for example, light with a wavelength of 660 nm) and infrared light (for example, light with a wavelength of 940 nm) are emitted on a body part, The absorbance of hemoglobin in blood for red light and infrared light varies whether or not the hemoglobin binds to oxygen. The recognition of an artery may be performed by known plethysmography. The blood index calculation unit 12 calculates the arterial oxygen saturation from the ratio of absorbed red light to absorbed infrared light (pulse spectrophotometry). Incidentally, a detailed calculation method of the arterial oxygen saturation may be basically the same as the method described in Non-Patent Document 1 (“Pulse Oximeter no Tanjo to Riron (Advent and Theory of Pulse Oximeter)”, written by Takuo Aoyagi, The Journal of Japan Society for Clinical Anesthesia, vol. 10, No. 1, pp. 1-11, 1990) or Non-Patent Document 2 (“ME Hayawakari (Quick Understanding) Q&A, Sphygmomanometer, Cardiac Output Meter, Blood Flow Meter” supervised by Yasuhisa Sakurai, Nankodo Co., Ltd.).

Further, the blood index calculation unit 12 calculates the amount of hemoglobin in blood using pulse spectrophotometry. The pulse spectrophotometry is a method of determining the ratios of the concentrations of substances in arterial blood by irradiating a biological tissue with light and measuring the light absorption property of arterial blood in the tissue utilizing the fact that the effective thickness of blood is pulsated by blood pulsation in the biological tissue. Various types of hemoglobin in blood (for example, oxygenated hemoglobin, deoxygenated hemoglobin, carboxyhemoglobin, and methemoglobin) and water have different light absorption properties, and therefore, by using light with a plurality of wavelengths, the ratios of the concentrations of various types of hemoglobin and water can be obtained. By calculating the ratio (CHb/Cw=Hb (g/ml)) of the sum of the ratios of the concentrations of various types of hemoglobin (CHb (g %)) with respect to the ratio of the concentration of water (Cw (g %)), the concentration of hemoglobin can be calculated. Incidentally, in this calculation, the volume of 1 g of water is assumed to be 1 ml. Therefore, in the case where the amount of hemoglobin in blood is calculated, it is necessary to irradiate a body part with light with a wavelength which is absorbed by water, Please see Patent Document 4 for a detailed calculation method of the amount of hemoglobin in blood using pulse spectrophotometry.

It is more preferred that the blood index calculation unit 12 calculates the amount of hemoglobin in blood, in which only hemoglobin capable of transferring oxygen (normal hemoglobin) is targeted (in other words, abnormal hemoglobin is excluded). That is, it is desired that the fractional oxygen saturation which is the ratio of oxygenated hemoglobin capable of transferring oxygen to the total hemoglobin is used. Alternatively, the concentration of oxygenated hemoglobin HbO2 (g/dl) may be directly calculated by calculating the ratio of the concentration of water Cw (g %) with respect to the ratio of the concentration of the above-mentioned oxygenated hemoglobin CHO2 (g %).

The abnormal hemoglobin refers to hemoglobin which does not have an ability to transfer oxygen, and is, for example, carboxyhemoglobin or methemoglobin.

In the case where the concentration of carboxyhemoglobin or the concentration of methemoglobin is calculated, it is only necessary to allow the light-emitting element 21 to irradiate a test subject with orange light (or red-orange light) in addition to near infrared light and red light as light to be irradiated onto the test subject. The transmitted light (or the reflected light) of each light changes in response to blood pulsation. The blood index calculation unit 12 determines an extinction ratio between respective wavelengths, and the concentration of carboxyhemoglobin or the concentration of methemoglobin may be calculated based on the extinction ratio. Please see JP-A-2002-228579 (Patent Document 6) which is the earlier application filed by the present inventors for a detailed calculation method of the concentration of carboxyhemoglobin. Similarly, please see JP-A-2002-315739 (Patent Document 7) which is the earlier application filed by the present inventors for a detailed calculation method of the concentration of methemoglobin. Since the concentration of abnormal hemoglobin can be calculated, the amount of normal hemoglobin can be calculated by multiplying the total amount of hemoglobin by the concentration of normal hemoglobin.

The blood index calculation unit 12 inputs the calculated arterial oxygen saturation and the calculated hemoglobin amount in blood to the SvO2 estimation unit 15. As described above, the blood index calculation unit 12 may input the total amount of hemoglobin in blood to the SvO2 estimation unit 15 as the amount of hemoglobin in blood, however, it is more preferred that a value (the amount of normal hemoglobin) obtained by subtracting the amount of abnormal hemoglobin from the total amount of hemoglobin in blood is input to the SvO2 estimation unit 15 as the amount of hemoglobin in blood.

The cardiac output calculation unit 13 calculates a cardiac output based on the biological signal obtained from a test subject. This will be described in detail below.

The cardiac output calculation unit 13 obtained an electrocardiographic signal from the electrocardiographic electrode 30 and calculates an electrocardiogram (ECG) from the electrocardiographic signal. The cardiac output calculation unit 13 calculates a heart rate (FIR) based on the waveform of the electrocardiogram. For example, the cardiac output calculation unit 13 may calculate a heart rate from the number of detected R waves.

Further, the cardiac output calculation unit 13 obtained a photoplethysmographic signal from the probe 20. As described above, the probe 20 performs light emission for measuring a general arterial oxygen saturation (SaO2). The cardiac output calculation unit 13 calculates a pulse wave transition time (PWTT) based on a photoplethysmographic waveform obtained from the photoplethysmographic signal and the electrocardiogram. Please see, for example, Non-Patent Document 3 (Internet <URL: http://www.nihonkohden.co.jp/iryo/techinfo/pwtt/principle.html> searched on Feb. 13, 2016) for the relationship among the photoplethysmographic waveform, the electrocardiogram, and the pulse wave transition time. Further, as for the detailed calculation method of the pulse wave transition time, the calculation may be performed by utilizing a pulse pressure, and for example, the same method as described in Patent Document 5 may be adopted.

The cardiac output calculation unit 13 estimates a cardiac output by substituting the pulse wave transition time and the heart rate in the following formula (1).

CO=(αL×PWTT+βL)×HR Formula (1)

In the formula (1), CO represents a cardiac output, PWTT represents a pulse wave transition time, HR represents a heart rate, and α, β, and L each represent a coefficient intrinsic to a test subject.

The respective coefficients in the above formula (1) are coefficients intrinsic to a test subject, however, these coefficients may be calibrated using calibration values obtained by the measurement of a blood pressure for calibration. As for a detailed calibration method, a method equivalent to the method described in Patent Document 5 (for example, FIG. 12 in Patent Document 5) may be adopted.

The cardiac output calculation unit 13 inputs the calculated cardiac output to the

SvO2 estimation unit 15. When using the above formula (1), the cardiac output can be continuously calculated. Therefore, it is preferred that the cardiac output calculation unit 13 continuously calculates the cardiac output and inputs the calculated cardiac output to the SvO2 estimation unit 15.

To the oxygen consumption calculation unit 14, a gas concentration in the expired air, a gas concentration in the inspired air, and a ventilation amount of a test subject are input from the respiration sensor 40. The oxygen consumption calculation unit 14 calculates a difference between the oxygen concentration in the gas concentration in the expired air and the oxygen concentration in the gas concentration in the inspired air (difference in oxygen concentration). The oxygen consumption calculation unit 14 calculates the oxygen consumption (VO2) of the test subject by multiplying the calculated difference in oxygen concentration by the ventilation amount. The oxygen consumption calculation unit 14 inputs the calculated oxygen consumption to the SvO2 estimation unit 15.

The SvO2 estimation unit 15 estimates the mixed venous oxygen saturation based on the amount of hemoglobin in blood, the arterial oxygen saturation, the oxygen consumption, and the cardiac output. The mechanism of this estimation will be described below.

It has been widely known that the Fick's formula (the following formula (2)) based on the law of blood circulation (the law of conservation of mass) is established (Non-Patent Document 4 (Yung GL, et.al, “Comparison of impedance cardiography to direct Fick and thermodilution cardiac output determination in pulmonary arterial hypertension.”Congestive Heart Failure 2004, 10 (2, suppl. 2): 7-10), Internet <URL: http://www.ncbi.nlm.nih,gov/pubmed/15073478> searched on Feb. 13, 2016)),

(CaO2−CvO2)×CO=VO2 Formula (2)

In the formula (2), CaO2 represents an arterial oxygen content, CvO2 represents a mixed venous oxygen content, CO represents a cardiac output, and VO2 represents an oxygen consumption.

Further, it is known that the amount of oxygen binding to 1 g of hemoglobin is from about 1.3 ml to 1.4 ml (Non-Patent Document 4). When this oxygen binding amount per gram of hemoglobin is represented by K (K is a constant between 1.3 and 1.4, preferably between 1.31 and 1.36. In general, K=1.34 is adopted), a difference between the arterial oxygen content and the mixed venous oxygen content, that is, the value of (CaO2−CvO2) can be represented by the following formula (3).

(CaO2−CvO2)=K×Hb×(SaO2−SvO2) Formula (3)

In the formula (3), K represents an oxygen binding amount per unit hemoglobin (1 g of hemoglobin), Hb represents the amount of hemoglobin in blood, SaO2 represents an arterial oxygen saturation, and SvO2 represents a mixed venous oxygen saturation. Here, SaO2 and SvO2 are each a fractional oxygen saturation. Abnormal hemoglobin (carboxyhemoglobin or methemoglobin) is hemoglobin which cannot bind to oxygen. In the case where the ratio of abnormal hemoglobin is large, when the functional oxygen saturation (the amount of oxygenated hemoglobin to the sum of the amount of oxygenated hemoglobin and the amount of deoxygenated hemoglobin) is substituted in the formula (3), CaO2 or CvO2, which is a blood oxygen content, becomes a value having an error. Due to this, in the case where the amount of abnormal hemoglobin is small, the functional oxygen saturation may be used in the formula (3), however, in the case where the amount of abnormal hemoglobin is large, it is not preferred to use the functional oxygen saturation in the formula (3).

Therefore, the mixed venous oxygen saturation can be calculated based on the formula 4) modified from the above formula (2) and formula (3). That is, the mixed venous oxygen saturation can be estimated from the calculation formula (formula (4)) based on a formula in Fick principle (a principle of blood circulation) and the oxygen binding amount per unit hemoglobin (for example, 1 g of hemoglobin).

SvO2 =SaO2−(VO2/(K×Hb×CO)) Formula (4)

In the formula (4), SvO2 represents a mixed venous oxygen saturation, SaO2 represents an arterial oxygen saturation, VO2 represents an oxygen consumption, Hb represents the amount of hemoglobin in blood, CO represents a cardiac output, and K represents an oxygen binding amount per unit hemoglobin (for example, 1 g).

The SvO2 estimation unit 15 calculates an estimation value of the mixed venous oxygen saturation by substituting the arterial oxygen saturation, the amount of hemoglobin in blood, the cardiac output, and the oxygen consumption calculated by the respective calculation units (the blood index calculation unit 12, the cardiac output calculation unit 13, and the oxygen consumption calculation unit 14) in the formula 4). The SvO2 estimation unit 15 inputs the calculated estimation value of the mixed venous oxygen saturation to the output unit 16.

The output unit 16 outputs various biological waveforms or the measurement values of various vital signs of a test subject. Here, the “output” may be a display output on a display or may be an output on a paper by printing. Further, the “output” is a concept including an output of an alarm sound when any of the vital signs is abnormal. Therefore, the output unit 16 is a display (and a peripheral circuit of the display), a printer (and a peripheral circuit thereof), a speaker (and a peripheral circuit thereof), or the like provided for the biological information measurement device 1. The output unit 16 outputs the mixed venous oxygen saturation estimated by the SvO2 estimation unit 15. For example, the output unit 16 displays a measurement value or a trend graph of the mixed venous oxygen saturation on a display. The output unit 16 may also display a measurement value or a measurement waveform of a general vital sign (a blood pressure or a body temperature) on the display in addition thereto.

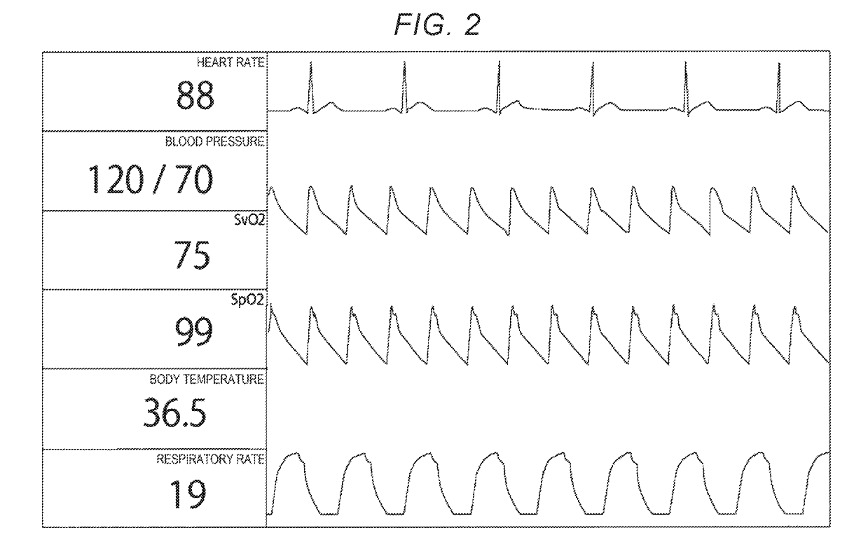

FIG. 2 is a view showing one example of a display screen on which the output unit 16 performs displaying. As shown in the drawing, on the display screen, an estimated value of the mixed venous oxygen saturation (SvO2) is displayed along with a heart rate and a blood pressure. The display screen shown in FIG. 2 is merely an example, and the estimated value of the mixed venous oxygen saturation may not only be displayed by a numerical value, but also may be displayed by a trend graph (a form by which a change over time is found).

Next, an effect of the biological information measurement device 1 according to this embodiment will be described. As described above, it is known that a blood index (an arterial oxygen saturation or the amount of hemoglobin in blood) associated with oxygen transport, an oxygen consumption, and a cardiac output can be noninvasively and accurately obtained by the function of a biological information monitor used in a general hospital. The SvO2 estimation unit 15 estimates a mixed venous oxygen saturation by substituting these parameters in a calculation formula derived from the blood circulation and the oxygen transport amount per unit hemoglobin. The SvO2 estimation unit 15 uses parameters which can be noninvasively and accurately calculated in the calculation, and therefore, the mixed venous oxygen saturation can be continuously and accurately estimated.

The techniques described in the above-mentioned Patent Documents 1 to 3 each require an exclusive sensor (in the technique described in patent Document 1, a light-emitting element for deep blood vessels, etc., in the technique described in patent Document 2, a photoacoustic imaging sensor, and in the technique described in patent Document 3, an MRS sensor). Due to this, these techniques have problems that the configuration of the device is complicated, and also the cost is high. Further, it is necessary to apply light to a blood vessel to be measured from immediately above the vessel, and therefore, more accurate measurement may not be able to be performed due to a lack of experience of a medical worker or the like.

On the other hand, the biological information measurement device 1 according to this embodiment can estimate the mixed venous oxygen saturation (SvO2) by the configuration of a biological information monitor using a common probe 20 for multiwavelength measurement, an electrocardiographic electrode 30, and a respiration sensor 40. That is, the mixed venous oxygen saturation can be estimated while avoiding the complication of the device or the increase in cost due to the adoption of an exclusive sensor. Further, the above-mentioned parameters can be noninvasively and accurately measured regardless of the experience or skill of a medical worker. Therefore, even if any medical worker is in charge of the test subject, the mixed venous oxygen saturation can be accurately estimated.

The transmitted light or the reflected light received by the probe 20 can also be used for the calculation of the amount of hemoglobin in blood along with the calculation of the arterial oxygen saturation. The transmitted light or the reflected light can be used in common for the calculation of a plurality of parameters, and therefore, the device can be simplified.

It is preferred that the blood index calculation unit 12 calculates a value obtained by subtracting the amount of abnormal hemoglobin from the total amount of hemoglobin in blood (in other words, only the amount of normal hemoglobin) as the amount of hemoglobin in blood. According to this, the effect of abnormal hemoglobin which cannot transport oxygen can be cancelled, and thus, the mixed venous oxygen saturation can be more accurately estimated.

Hereinabove, the invention made by the present inventors has been specifically described based on embodiments, however, the invention is not limited to the above-mentioned embodiments, and it is needless to say that various modifications can be made without departing from the gist of the invention.

The calculation methods of the respective parameters described above are merely examples, and other methods may be used. That is, the SvO2 estimation unit 15 may estimate the mixed venous oxygen saturation by substituting the measurement values of the respective parameters obtained by an arbitrary method which is noninvasive and is not affected by the experience of a medical worker in the above-mentioned calculation formula.

For example, the amount of hemoglobin in blood may be measured from blood collected in advance from a test subject. In this case, it is only necessary to adopt a configuration in which the probe 20 or the blood index calculation unit 12 calculates only the arterial oxygen saturation (that is, a general configuration in which the measurement of an arterial oxygen saturation is performed).

Further, the cardiac output calculation unit 13 may calculate the cardiac output from a blood flow signal as described in Patent Document 8 (JP-A-2003-220045) or Non-Patent Document 5 (Internet <URL: http://www.kykb.jp/manatec1.html> searched on Feb. 13, 2016). Further, the cardiac output calculation unit 13 may calculate the cardiac output noninvasively based on the value of a blood pressure measured from a finger or the like (Patent Document 9 (JP-T-2002-541961) or Non-Patent Document 6 (Internet <URL: http://www.edwards.com/jp/professionals/products/hemodynamic_monitoring/co_sv/clears ight/#> searched on Feb. 13, 2016)). The cardiac output calculation unit 13 may also calculate the cardiac output noninvasively using a method called a so-called “reactance method” (Patent Document 10 (JP-T-2014-521433) or Non-Patent Document 7 (Internet <URL: http://www.imimed.co.jp/product/monitor/detail/starling_sv.html> searched on Feb. 13, 2016)). Further, as described in Patent Document 11 (JP-A-2006-231012), an oxygen circulation time is measured, and an estimation value of the cardiac output correlated with the oxygen circulation time may be calculated using a regression equation.

That is, the cardiac output calculation unit 13 may calculate the cardiac output based on a biological signal obtained from a test subject using a noninvasive method.

At least part of the processing performed by the above-mentioned various processing units (the light emission control unit 11, the blood index calculation unit 12, the cardiac output calculation unit 13, the oxygen consumption calculation unit 14, and the SvO2 estimation unit 15) may be realized by causing a CPU (Central Processing Unit, not shown in FIG. 1) provided in the biological information measurement device 1 to execute a program.

Here, the program is stored using various types of non-transitory computer readable media and can be supplied to a computer. The non-transitory computer readable media include various types of tangible storage media. Examples of the non-transitory computer readable media include magnetic recording media (such as flexible disks, magnetic tapes, and hard disk drives), magneto-optical recording media (such as magneto-optical disks), CD-ROM (Read Only Memory), CD-R, CD-R/W, semiconductor memories (such as mask ROM, PROM (Programmable ROM), EPROM (Erasable PROM), flash ROM, and RAM (random access memory). Further, the program may be supplied to a computer through any of various types of transitory computer readable media. Examples of the transitory computer readable media include electrical signals, optical signals, and electromagnetic waves. The transitory computer readable media can supply the program to a computer through a wired communication channel such as an electrical wire or an optical fiber or a wireless communication channel

What is claimed is:

1. A biological information measurement device, comprising:
a blood index calculation unit configured to calculate at least part of a blood index associated with oxygen transport based on transmitted light or reflected light when a body part of a test subject is irradiated with light with a plurality of wavelengths;
an oxygen consumption calculation unit configured to calculate an oxygen consumption based on a gas concentration in inspired air, a gas concentration in expired air, and a ventilation amount of the test subject;
a cardiac output calculation unit configured to calculate a cardiac output based on a biological signal obtained by a noninvasive method from the test subject; and
an SvO2 estimation unit configured to estimate a mixed venous oxygen saturation of the test subject by substituting the blood index, the oxygen consumption, and the cardiac output in a calculation formula derived from a principle of blood circulation and an oxygen binding amount per unit hemoglobin,

2. The biological information measurement device according to claim 1, wherein the calculation formula is represented by the following formula:
SvO2=SaO2−(VO2/(K×Hb×CO)) Formula (4)
wherein SvO2 represents the mixed venous oxygen saturation, SaO2 represents an arterial oxygen saturation, VO2 represents the oxygen consumption, Hb represents an amount of hemoglobin in blood, CO represents the cardiac output, and K represents the oxygen binding amount per unit hemoglobin (for example, 1 g).

3. The biological information measurement device according to claim 2, wherein the K is a value between 1.3 and 1.4.

4. The biological information measurement device according to claim 1, further comprising:
a light emission control unit configured to control the light with the plurality of wavelengths,
wherein the light with the plurality of wavelengths includes light which is absorbed by water, and
wherein the blood index calculation unit is configured to calculate an amount of hemoglobin in blood along with an arterial oxygen saturation based on the transmitted light or the reflected light.

5. The biological information measurement device according to claim 2, further comprising:
a light emission control unit configured to control the light with the plurality of wavelengths,
wherein the light with the plurality of wavelengths includes light Which is absorbed by water, and
the blood index calculation unit is configured to calculate the amount of hemoglobin in blood along with the arterial oxygen saturation based on the transmitted light or the reflected light.

6. The biological information measurement device according to claim further comprising:
a light emission control unit configured to control the light with the plurality of wavelengths,
wherein the light with the plurality of wavelengths includes light which is absorbed by water, and
the blood index calculation unit is configured to calculate the amount of hemoglobin in blood along with the arterial oxygen saturation based on the transmitted light or the reflected

7. The biological information measurement device according to claim 1,
wherein the blood index calculation unit is configured to subtract an amount of abnormal hemoglobin from a total amount of hemoglobin in the blood of the test subject as an amount of hemoglobin in blood as the at least part of the blood index.

8. The biological information measurement device according to claim 2,
wherein the blood index calculation unit is configured to subtract an amount of abnormal hemoglobin from a total amount of hemoglobin in the blood of the test subject as the amount of hemoglobin in blood as the at least part of the blood index.

9. The biological information measurement device according to claim 3,
wherein the blood index calculation unit is configured to subtract an amount of abnormal hemoglobin from a total amount of hemoglobin in the blood of the test subject as the amount of hemoglobin in blood as the at least part of the blood index.

10. The biological information measurement device according to claim 4,
wherein the blood index calculation unit is configured to subtract an amount of abnormal hemoglobin from a total amount of hemoglobin in the blood of the test subject as the amount of hemoglobin in blood as the at least part of the blood index.

11. The biological information measurement device according to claim 5,
wherein the blood index calculation unit is configured to subtract an amount of abnormal hemoglobin from a total amount of hemoglobin in the blood of the test subject as the amount of hemoglobin in blood as the at least part of the blood index.

12. The biological information measurement device according to claim 6,
wherein the blood index calculation unit is configured to subtract an amount of abnormal hemoglobin from a total amount of hemoglobin in the blood of the test subject as the amount of hemoglobin in blood to obtain the at least part of the blood index.

13. A mixed venous oxygen saturation estimation method, comprising:
calculating at least part of a blood index associated with oxygen transport based on transmitted light or reflected light when a body part of a test subject is irradiated with light with a plurality of wavelengths;
calculating an oxygen consumption based on a gas concentration in inspired air, a gas concentration in expired air, and a ventilation amount of the test subject;
calculating a cardiac output based on a biological signal obtained by a noninvasive method from the test subject; and
estimating the mixed venous oxygen saturation of the test subject by substituting the blood index, the oxygen consumption, and the cardiac output in a calculation formula derived from a principle of blood circulation and an oxygen binding amount per unit hemoglobin.

14. The method of claim 13, further comprising calculating an amount of hemoglobin in blood along with an arterial oxygen saturation based on the transmitted light or the reflected light,
wherein the light with a plurality of wavelengths includes light which is absorbed by water.

15. The method of claim 13, further comprising:
subtracting an amount of abnormal hemoglobin from a total amount of hemoglobin in the blood of the test subject as an amount of hemoglobin in blood as the at least part of the blood index,

16. A non-transitory computer readable medium comprising instructions that, when executed by one or more processing units, cause the one or more processing units to perform actions including:
calculating at least part of a blood index associated with oxygen transport based on transmitted light or reflected light when a body part of a test subject is irradiated with light with a plurality of wavelengths,
calculating an oxygen consumption based on a gas concentration in inspired air, a gas concentration in expired air, and a ventilation amount of the test subject,
calculating a cardiac output based on a biological signal obtained by a noninvasive method from the test subject, and
estimating the mixed venous oxygen saturation of the test subject by substituting the blood index, the oxygen consumption, and the cardiac output in a calculation formula derived from a principle of blood circulation and an oxygen binding amount per unit hemoglobin.

17. The non-transitory computer-readable medium of claim 10, wherein the actions further includes calculating an amount of hemoglobin in blood along with an arterial oxygen saturation based on the transmitted light or the reflected light,
wherein the light with a plurality of wavelengths includes light which is absorbed by water.

18. The non-transitory computer-readable medium of claim 10, wherein the actions further includes subtracting an amount of abnormal hemoglobin from a total amount of hemoglobin in the blood of the test subject as an amount of hemoglobin in blood as the at least part of the blood index.
